(19) United States
(12) Patent Application Publication
(10) Pub. No.: US 2023/0329374 A1
(43) Pub. Date: Oct. 19, 2023

(54) Disinfecting Glove Device

(71) Applicants: Kurt Williams, Indianapolis, IN (US); Nicholas Williams, Indianpolis, IN (US)

(72) Inventors: Kurt Williams, Indianapolis, IN (US); Nicholas Williams, Indianpolis, IN (US)

(21) Appl. No.: 18/191,345

(22) Filed: Mar. 28, 2023

Related U.S. Application Data
(60) Provisional application No. 63/330,985, filed on Apr. 14, 2022.

(51) Int. Cl. A41D 19/00; A61L 2/10

(52) U.S. Cl. CPC A41D 19/0024; A61L 2/10

(57) ABSTRACT

The present invention relates to a disinfecting glove device comprised of a glove body with at least one UV light. The UV light is positioned on the inside surface of the glove body. The UV light preferably emits UV light in the frequency of 150-230 nanometers during use. As a result, germs, bacteria, and viruses on surfaces touched by the inside surface of the device are killed via the UV light.

### CROSS-REFERENCE TO RELATED APPLICATION

The present application claims priority to, and the benefit of, U.S. Provisional Application No. 63/330,985, which was filed on Apr. 14, 2022, and is incorporated herein by reference in its entirety.

### FIELD OF THE INVENTION

The present invention relates generally to the field of gloves. More specifically, the present invention relates to a disinfecting glove device comprised of a glove body with at least one UV light. The UV light is positioned on the inside surface of the glove body. The UV light preferably emits UV light in the frequency of 150-230 nanometers during use. As a result, germs, bacteria, and viruses on surfaces touched by the inside surface of the device are killed via the UV light. Accordingly, the present disclosure makes specific reference thereto. Nonetheless, it is to be appreciated that aspects of the present invention are also equally applicable to other like applications, devices, and methods of manufacture.

### BACKGROUND

Germs, viruses, and bacteria are highly contagious and easily spread from person to person via unclean hands touching various surfaces. Individuals often touch said surfaces and may spread or contract serious illnesses as a result. Without constant sanitization of doorknobs, elevator buttons, door handles, and numerous other public surfaces, people will constantly spread harmful germs, viruses, and bacteria.

Therefore, there exists a long-felt need in the art for an improved disinfecting device. There also exists a long-felt need in the art for a disinfecting glove device that can be worn while touching public surfaces. More specifically, there exists a long-felt need in the art for a disinfecting glove device that disinfects surfaces touched by the device. In addition, there exists a long-felt need in the art for a disinfecting glove device that disinfects surfaces touched by the device, wherein the device disinfects surfaces in an efficient manner.

The subject matter disclosed and claimed herein, in one embodiment thereof, comprises a disinfecting glove device. The device is comprised of a glove body with at least one UV light. The UV light is positioned on the inside surface of the glove body. The UV light preferably emits UV light in the frequency of 150-230 nanometers during use. As a result, germs, bacteria, and viruses on surfaces touched by the inside surface of the device are killed via the UV light. The UV light can be turned on/off via at least one control mechanism located on the device. The UV light is powered by at least one battery stored within a battery pack.

In this manner, the disinfecting glove device of the present invention accomplishes all the foregoing objectives and provides an improved disinfecting device that can be worn while touching public surfaces. More specifically, the device disinfects surfaces touched by the device using the UV light of the device. In doing so, the device disinfects surfaces in an efficient manner.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a disinfecting glove device comprised of a glove body with at least one UV light. The body is preferably shaped such that it can accommodate a hand of a user and may be made from any glove material known in the art. The material is preferably non-penetrable to eliminate the possibility of the body becoming punctured by a foreign object such that germs, bacteria, and viruses can enter the body.

The wrist area of the body is comprised of at least one closure. The closure allows a user to tighten the device on their wrist. The closure prevents germs and other debris from entering the body at the wrist area.

The inside surface of the body may be comprised of any material that promotes grip. The inside surface may also be comprised of at least one texture. The texture improves the grip of the inside surface while touching surfaces.

Each finger and/or palm area on the inside surface is comprised of at least one UV light. In one embodiment, the UV light is exposed on the inside surface via at least one opening that forms an air-tight seal around the UV light to prevent germs, bacteria, viruses, etc., from entering the body via the opening. In a different embodiment, the UV light shines through the inside surface.

The UV light may be any UV light known in the art with a nanometer frequency between 150-230 nanometers. As a result, the UV light prevents the spread of germs, bacteria, and viruses on the device and surfaces the device touches by killing the germs, bacteria, and viruses using the UV light as the user's hand touches the surface while wearing the device.

The device is also comprised of a battery stored within a battery pack on the outside of the body. The battery is in wired electrical communication with each light via at least one wire. The wire is preferably comprised of a flexible, lightweight conduit. The wire is preferably positioned within an inner layer of the body material. The device is also comprised of at least one switch. The switch may be in the form of a switch, toggle, button, etc., that allows the user to power on/off the UV light.

Accordingly, the disinfecting glove device of the present invention is particularly advantageous as it provides an improved disinfecting device that can be worn while touching public surfaces. More specifically, the device disinfects surfaces touched by the device using the UV light of the device. In doing so, the device disinfects surfaces in an efficient manner. In this manner, the disinfecting glove device provides a novel solution to disinfecting surfaces.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front and rear perspective view of one potential embodiment of a disinfecting glove device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for an improved disinfecting device. There also exists a long-felt need in the art for a disinfecting glove device that can be worn while touching public surfaces. More specifically, there exists a long-felt need in the art for a disinfecting glove device that disinfects surfaces touched by the device. In addition, there exists a long-felt need in the art for a disinfecting glove device that disinfects surfaces touched by the device, wherein the device disinfects surfaces in an efficient manner.

The present invention, in one exemplary embodiment, is comprised of a disinfecting glove device comprised of a glove body with at least one UV light. The body is preferably shaped such that it can accommodate a hand of a user and may be made from any glove material known in the art. The material is preferably non-penetrable to eliminate the possibility of the body becoming punctured by a foreign object such that germs, bacteria, and viruses can enter the body.

The wrist area of the body is comprised of at least one closure. The closure allows a user to tighten the device on their wrist. The closure also prevents germs and other debris from entering the body at the wrist area.

Furthermore, the inside surface of the body may be comprised of any material that promotes grip. The inside surface may also be comprised of at least one texture, wherein the texture improves the grip of the inside surface while touching surfaces. Each finger and/or palm area on the inside surface is comprised of at least one UV light. In one embodiment, the UV light is exposed on the inside surface via at least one opening that forms an air-tight seal around the UV light to prevent germs, bacteria, viruses, etc., from entering the body via the opening. In a different embodiment, the UV light shines through the inside surface.

The UV light may be any UV light known in the art with a nanometer frequency between 150-230 nanometers. As a result, the UV light prevents the spread of germs, bacteria, and viruses on the device and surfaces the device touches by killing the germs, bacteria, and viruses using the UV light as the user's hand touches the surface while wearing the device.

The device is also comprised of a battery. The battery is stored within a battery pack on the outside of the body. The battery is in wired electrical communication with each light via at least one wire. The wire is preferably comprised of a flexible, lightweight conduit. The wire is preferably positioned within an inner layer of the body material. The device is also comprised of at least one switch. The switch may be in the form of a switch, toggle, button, etc., that allows the user to power on/off the UV light.

Accordingly, the disinfecting glove device of the present invention is particularly advantageous as it provides an improved disinfecting device that can be worn while touching public surfaces. More specifically, the device disinfects surfaces touched by the device using the UV light of the device. In doing so, the device disinfects surfaces in an efficient manner. In this manner, the disinfecting glove device provides a novel solution to disinfecting surfaces.

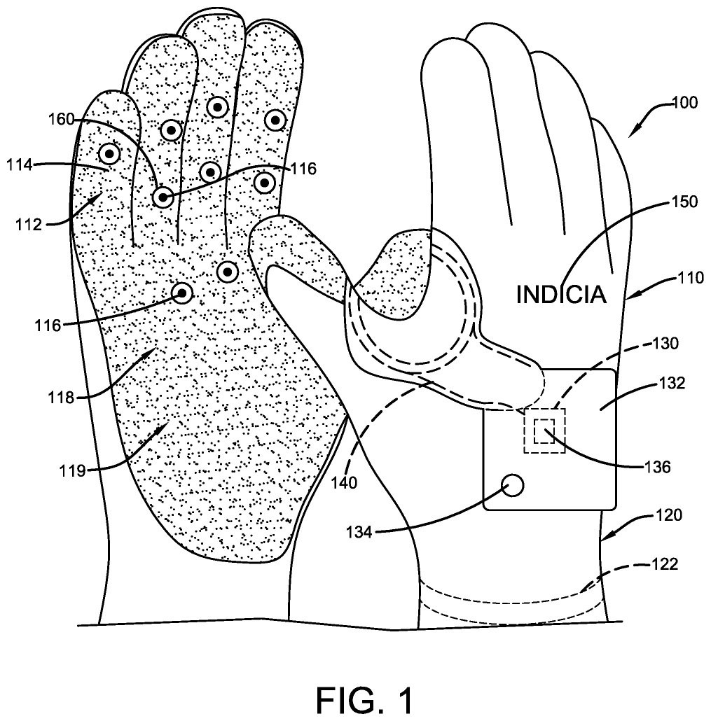

Referring initially to the drawings, FIG. 1 illustrates a front and rear perspective view of one potential embodiment of a disinfecting glove device 100 of the present invention in accordance with the disclosed architecture. The device 100 is comprised of a glove body 110 with at least one UV light 116. The body 110 is preferably shaped such that it can accommodate a hand of a user. The body 110 preferably has at least one finger 112 that can accommodate a user's finger/thumb. In another embodiment, the finger 112 can accommodate all of a user's fingers, wherein the device 100 is a mitten-style garment. In the preferred embodiment, the body 110 has five fingers 112 to accommodate a user's thumb, index finger, middle finger, ring finger, and pinky finger.

The body 110 may be any color known in the art. The body 110 may be comprised of at least one indicia 150. The indicia 150 includes patterns, logos, emblems, images, symbols, designs, letters, numbers, words, characters, animals, advertisements, brands, etc.

The body 110 may be made from any glove material known in the art. This includes, but is not limited to, polyethylene, high-performance polyethylene, leather, synthetic material, neoprene, nitrile, polyurethan, latex, polyvinyl chloride, etc. The material may be an insulated or non-insulated material. The material is preferably non-penetrable to eliminate the possibility of the body 110 becoming punctured by a foreign object such that germs, bacteria, and viruses can enter the body 110.

The wrist area 120 of the body 110 is comprised of at least one closure 122. The closure allows a user to tighten the device 100 on their wrist. In differing embodiments, the closure 122 may be any closure type known in the art such as, but not limited to, an elastic band, a fastener, etc. The closure 122 prevents germs and other debris from entering the body 110 at the wrist area.

The inside surface 114 of the body 110 may be comprised of any material that promotes grip. This includes, but is not limited to, synthetic leather, silicone, nitrile, polyvinyl chloride, etc. In this manner, the device 100 does not slip off surfaces during use. The inside surface material is preferably a non-slip material.

The inside surface 114 may also be comprised of at least one texture 119. The texture 119 may be a raised or recessed texture. The texture 119 improves the grip of the inside surface 114 while touching surfaces.

Each finger 112 and/or palm area 118 on the inside surface 114 is comprised of at least one UV light 116. The UV light 116 is exposed on the inside surface 114 via at least one opening 160. In one embodiment, the opening 160 forms an air-tight seal around the UV light 116 to prevent germs, bacteria, viruses, etc., from entering the body 110 via the opening 160. In a different embodiment, the UV light 116 shines through the inside surface 114.

The UV light 116 may be any UV light known in the art with a nanometer frequency between 150-230 nanometers. As a result, the UV light prevents the spread of germs, bacteria, and viruses on the device 100 and surfaces the device 100 touches by killing the germs, bacteria, and viruses using the UV light 116 as the user's hand touches the surface while wearing the device 100.

The light 116 is powered by at least one battery 130. The battery 130 may be a disposable battery 130 or a rechargeable battery 130 in the form of an alkaline, nickel-cadmium, nickel-metal hydride battery 130, etc., such as any 3V-12 volts DC battery 130 or other conventional battery 130 such as A, AA, AAA, etc., that supplies power to the device 100. Throughout this specification, the terms “battery” and “batteries” may be used interchangeably to refer to one or more wet or dry cells or batteries 130 of cells in which chemical energy is converted into electricity and used as a source of DC power. References to recharging or replacing batteries 130 may refer to recharging or replacing individual cells, individual batteries 130 of cells, or a package of multiple battery cells as is appropriate for any given battery 130 technology that may be used. Additionally, the battery 130 may be recharged by a USB port 136 (of any USB type known in the art).

In the preferred embodiment, the battery 130 is stored within a battery pack 132 on the outside of the body 110. The battery 130 is in wired electrical communication with each light 118 via at least one wire 140. The wire 140 is preferably comprised of a flexible, lightweight conduit. The wire 140 is preferably positioned within an inner layer of the body 110 material.

The device 100 is comprised of at least one control mechanism 134. The mechanism 134 may be in the form of a switch, toggle, button, etc., that allows the user to power on/off the UV light 116. The UV light 116 may be programmed to run for a specific period of time or until the light 116 is turned off by the switch 116. The switch 116 may be located on the outside or inside of the body 110.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “disinfecting glove device” and “device” are interchangeable and refer to the disinfecting glove device 100 of the present invention.

Notwithstanding the foregoing, the disinfecting glove device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the disinfecting glove device 100 as shown in the FIGS. are for illustrative purposes only, and that many other sizes and shapes of the disinfecting glove device 100 are well within the scope of the present disclosure. Although the dimensions of the disinfecting glove device 100 are important design parameters for user convenience, the disinfecting glove device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A disinfecting glove device comprising:
a glove body;
a UV light;
a battery; and
a control mechanism.

2. The disinfecting glove device of claim 1, wherein the UV light is comprised of a nanometer frequency between 150-230 nanometers.

3. The disinfecting glove device of claim 1, wherein the UV light can be turned on or off by the control mechanism.

4. The disinfecting glove device of claim 3, wherein the control mechanism is comprised of a switch or a button.

5. A disinfecting glove device comprising:
a glove body;
a UV light positioned on an inner surface of the glove body;
a battery positioned within a battery pack; and
a control mechanism in electrical communication with the UV light.

6. The disinfecting glove device of claim 5, wherein the glove body is comprised of a finger.

7. The disinfecting glove device of claim 5, wherein the UV light is comprised of a nanometer frequency between 150-230 nanometers.

8. The disinfecting glove device of claim 5, wherein the battery is removable from the battery pack.

9. The disinfecting glove device of claim 5, wherein the inner surface is comprised of a synthetic leather, a silicone, a nitrile, or a polyvinyl chloride material.

10. A disinfecting glove device comprising:
a glove body comprised of a textured inner surface;
a UV light positioned on the textured inner surface of the glove body;
a battery;
a charging port; and
a control mechanism in electrical communication with the UV light.

11. The disinfecting glove device of claim 10, wherein the UV light is comprised of a nanometer frequency between 150-230 nanometers.

12. The disinfecting glove device of claim 10, wherein the textured inner surface is comprised of a raised texture.

13. The disinfecting glove device of claim 10, wherein the textured inner surface is comprised of a recessed texture.

14. The disinfecting glove device of claim 10, wherein the charging port is comprised of a USB charging port.

15. The disinfecting glove device of claim 10, wherein the glove body is comprised of a closure positioned on a wrist area of the glove body.

16. The disinfecting glove device of claim 15, wherein the closure is comprised of an elastic band.

17. The disinfecting glove device of claim 15, wherein the closure is comprised of a fastener.

18. The disinfecting glove device of claim 10, wherein the body is comprised of a non-penetrable material.

19. The disinfecting glove device of claim 10, wherein the body is comprised of an insulating material.

20. The disinfecting glove device of claim 10, wherein the control mechanism allows a user to turn the UV light on or off.
